(19) United States
(12) Patent Application Publication
(10) Pub. No.: US 2006/0189989 A1
(43) Pub. Date: Aug. 24, 2006

(54) Zero profile template for installation of surgical plate

(76) Inventor: Jeffrey K. Bert, Coos Bay, OR (US)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 11/055,279

(22) Filed: Feb. 10, 2005

(51) Int. Cl. A61F 2/30

(52) U.S. Cl. 606/69

(57) ABSTRACT

A method and apparatus for safely creating a cavity within living bone into which a plate or implant can be embedded or countersunk. A template is attached to the bone in a required location and is used to control the working depth of a cutting tool used to remove bone within an area limited by the template into which the plate can be placed.

### BACKGROUND OF THE INVENTION

The present invention relates to spinal surgery and particularly to anterior cervical vertebral fusion. Spinal fusions are often necessary to resolve severe pain caused by a bulging or herniated disk. When a fusion is performed, the injured disk is identified and the disk and adjacent vertebrae are surgically exposed. The vertebrae are separated and the injured disk is removed from between them. A bone graft is then inserted between the vertebrae. A plate is then fastened to the vertebrae to interconnect and immobilize them until the fusion is complete.

A surgical plate is formed of rigid or semi-rigid biocompatible material, commonly metal such as stainless steel, titanium, or titanium alloy. A plate is typically fastened to the surface of bone using screws or other fasteners which extend through holes in the plate and into the bone. Plates can be permanent or may be designed to degrade or become absorbed over time. Plates may be used to immobilize sections of fractured bone to permit healing. For example, if a long bone such as a femur is fractured, a plate may be attached to the femur bridging the fracture in such a way that the fracture is held together by the plate and thus immobilized for healing.

When a fusion is performed to the cervical spine, the vertebrae are accessed surgically through the anterior neck. The disk is manipulated from the anterior of the spine, and the plate is attached to the anterior surface of the vertebrae. Because of the proximity of neck structures, including the esophagus, the profile of the plate, which can extend from the anterior surfaces of the vertebrae by as much as 2 mm, can interfere with the function of the neck structures. For example, dysphagia is present in the majority of patients during the first four to six months following a cervical spinal fusion. In some patients, the complication resolves itself, but in others it continues to cause discomfort for an extended period of time. One cause of the dysphagia is the profile of the plate, which protrudes from the spine and causes pressure on, or some degree of displacement of the esophagus.

Placement of a plate on the anterior side of the spine may be complicated by the structure of a vertebra. An individual vertebra is made up of several components which can dictate techniques for the safe placement of surgical plates. The body of the vertebra is the primary weight bearing structure and provides a resting place for the fibrous disks which separate the vertebrae from each other. The lamina covers the spinal canal, which is a large hole in the center of the vertebra through which the spinal nerves pass. Attachment of a plate should not compromise the integrity of the lamina or the spinal canal. If it does, then spinal injury may result. For this reason, penetration of the vertebrae, for example with cutters, drills, or screws, can be dangerous unless the method employed protects the integrity of the spinal structures by limiting the depth of penetration.

A method and apparatus for safely implanting a surgical plate, particularly in connection with creating a cervical spinal fusion which would not interfere with the function of the esophagus, is therefore desirable.

### BRIEF SUMMARY OF THE INVENTION

As one principal aspect of the present invention, a method is provided for installing an implant such as a plate or other device in at least one bone, the method including providing an implant structure having a predetermined shape and size to be installed, providing a template for guiding a cutter in three dimensions, placing the template on the bone or bones, and using the cutter to create a cavity the approximate shape and size of the implant structure to be installed, then installing the structure in the cavity.

In one preferred embodiment, a method of affixing a surgical plate to at least one cervical vertebra includes providing a plate having a length, a width, and a thickness, creating a cavity in bone having substantially the same shape and size as the plate, and installing the plate within said cavity.

As another aspect of the invention, in one preferred embodiment, an apparatus for removing bone in order to countersink a surgical plate includes a frame which defines an internal area and has a track associated with the frame for guiding a cutter in three dimensions to create a space substantially the same size as the plate.

The foregoing and other objectives, features, and advantages of the invention will be more readily understood upon consideration of the following detailed description of the invention, taken in conjunction with the accompanying drawings.

### BRIEF DESCRIPTION OF THE SEVERAL VIEWS OF THE DRAWINGS

FIG. 1 is an exploded view of a template which is one preferred embodiment of one aspect of the invention, together with cervical portion of a spinal column.

FIG. 2 is a perspective view of the device shown in FIG. 1 in use on a section of a cervical spine.

FIG. 3 is a perspective view of the spinal section of FIG. 2 showing a recess created by the apparatus shown in FIGS. 1 and 2.

FIG. 4 is a sectional view taken along line 4-4 of FIG. 2, showing the device in use.

FIG. 5 is a perspective view of the section of spine shown in FIGS. 2 and 3, with a plate countersunk in the recess formed using the device.

FIG. 6 is a sectional view similar to FIG. 4, showing use of an alternative embodiment of the apparatus shown in FIGS. 1-4.

### DETAILED DESCRIPTION OF PREFERRED EMBODIMENT

A method and apparatus are provided for safely countersinking a surgical plate or any other implant to be affixed to or imbedded in bone. This method and apparatus may be used to alleviate complications such as dysphagia following neck surgery, or may be used for the installation of a biological plate of material which is intended to fuse with the tissue to which it is attached and is embedded to promote or enhance such fusion.

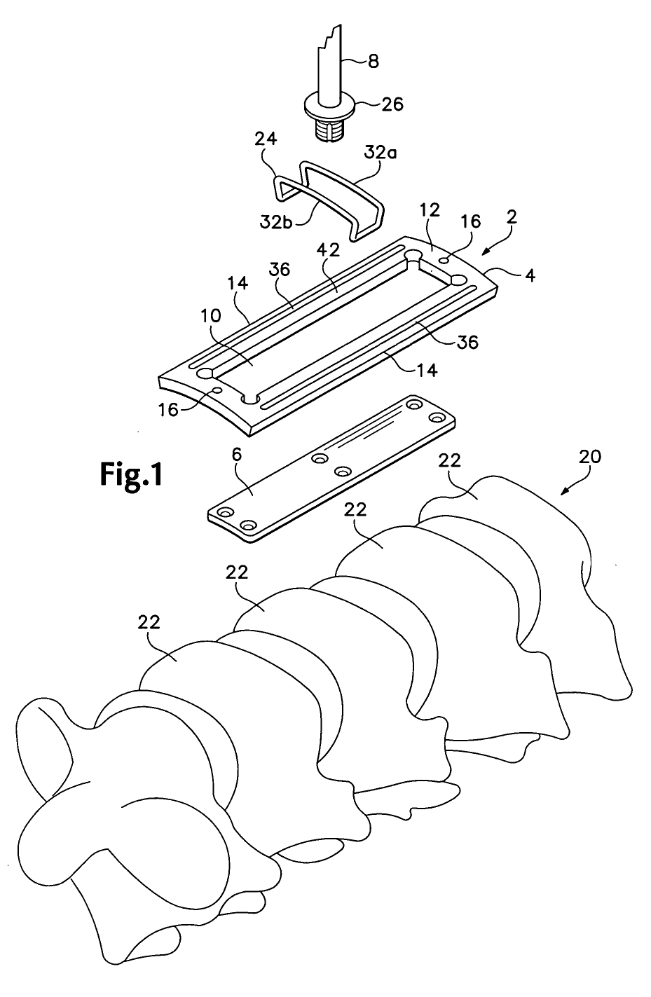

Referring first to FIGS. 1, 2, 3, and 4 of the drawings which form a part of the disclosure herein, the apparatus includes a combination of a cutting tool and a guiding apparatus referred to herein as a template 2, which includes a frame 4 that may be made of suitably formed metal and which defines an interior area 10 which may preferably be the approximate size and shape of the plate 6 or implant to be installed. The template also provides a way of controlling the depth 48 reachable by a cutter such as a burr 8 engaged with the template 2. The frame 4 may include a pair of end pieces 12 interconnecting a pair of side pieces 14. The frame 4 may be roughly rectangular, or of any other shape compatible with the plate 6 to be installed or capable of guiding the creation of a cavity which is a shape compatible with the plate to be installed. The frame 4 includes fastener receptacles 16 defined, for example, in the end pieces 12, which allow the surgeon to use fasteners 18 such as tacks or brads to temporarily but securely fasten the template 2 to the surface 20 of a bone or bones 22 in which the plate 6 will be installed.

When the surgeon is ready to install an implant or a surgical plate 6, the apparatus including the template 2 and a corresponding cutter such as the burr 8 are used to safely and effectively countersink the plate 6 according to the present method. First, radiographs or other images are used to identify the correct location for the plate 6 on the spinal column or other bone 22. The correct position of the plate 6 is indicated by therapeutic considerations. Next, the surgeon attaches the template 2 in the location selected. In order to attach the template 2 to the bone 22, the surgeon uses fasteners 18 such as tacks or brads which can be driven into the bone 22 using a mallet or hammer. When the template 2 is thus attached, additional radiographic or other images can be taken to confirm that it is in the correct location. Once the template 2 is attached and its position confirmed, it is used to safely guide a suitable corresponding cutter such as the burr 8 in removing material from the appropriate portion of the bone 22 in order to create a recess for at least partially countersunk placement of the plate 6 in the bone 22 so that it preferably has no protruding profile.

A surgeon uses a burr 8 or other cutter to remove a portion of the bone 22 within the internal area 10 with the burr 8 guided by the frame 4. The frame 4 defines an internal area 10 approximately the length and width of the plate to be installed, so that when a thin layer of bone as defined by the internal area 10 is removed, the bone is prepared for the installation of the surgical plate 6. The template 2 uses the frame 4 and a movable track set 24 to guide the burr 8 for removal of the bone.

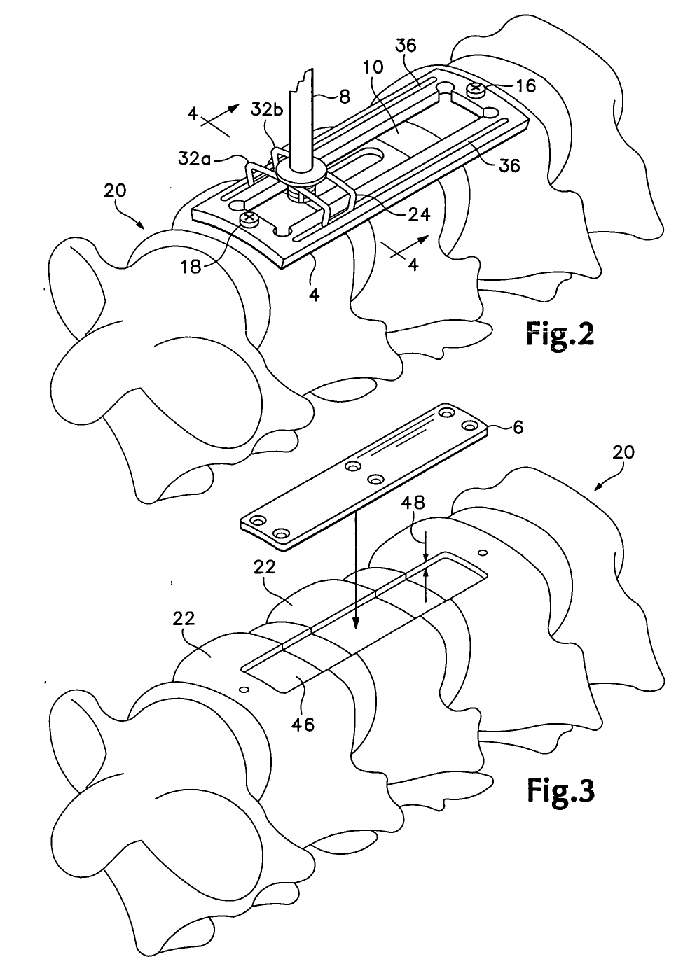

As shown in FIGS. 1, 2, and 4, the track set 24 may be a slotted rail such as an appropriately shaped loop of wire, or a small metal sheet defining as primary elements a pair of parallel rails such as guide wires 32a and 32b spaced apart widely enough to permit a cutting burr 8 to fit between them but located closely enough together so that a skirt 26 on the cutting burr 8 cannot fit between them. The track set 24 is moveably engaged with the frame 4, and when the cutter or burr 8 is engaged with the track set 24, the track set 24 guides its movement around the internal area 10, and limits the depth 48 reachable by the burr 8 (FIGS. 2-4) so that the burr is prevented from penetrating too far into the bone. Thus, use of the template 2 and the burr 8 allows for the safe removal of sufficient bone to countersink a plate 6 or an implant without serious related risk of harm to the patient.

The skirt 26 is fastened securely on the shaft 33 of the cutter or burr 8 and, in conjunction with the guide wires 32a, 32b, prevents the burr 8 from penetrating too deep. The guide wires 32a, 32b may be parallel to the end pieces 12 of the frame 4 and perpendicular to the side pieces 14. The track set 24 is slideably affixed to the frame 4. In one embodiment, the track set 24 is slideably affixed to the side pieces 14 of the frame 4. For example, the guide wires 32a, 32b may terminate in relatively large portions such as knobs 34 which fit slidably in compatibly shaped guide channels 36 in the side pieces 14. The guide channels 36 may be teardrop shaped, so that the channel is narrower at its opening slot 38, which is open into a long, narrow trough 40 that is somewhat wider than the slot 38. Thus, the knobs 34 slide freely within the channels 36 but are not easily removable from the channels 36 because they are larger in diameter than the width of the slots 38. Alternatively, the guide channels 36 could be simple grooves spaced apart an approximate distance to receive the opposite ends of the track set 24, allowing it to slide longitudinally along the frame 4.

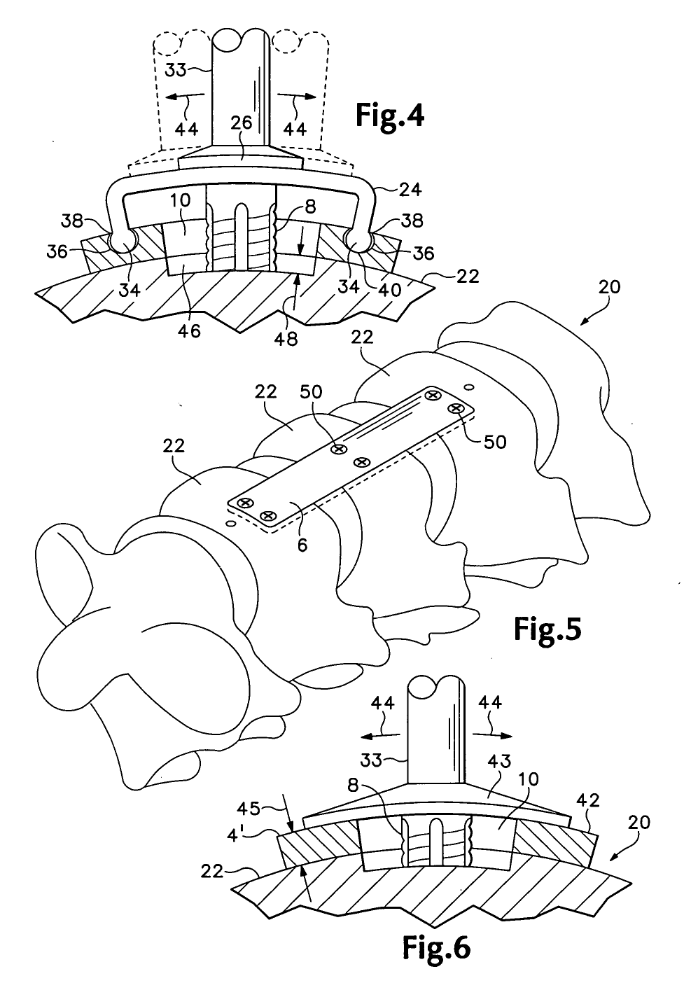

During use, the surgeon inserts the burr 8 between the guide wires 32a, 32b. The surgeon removes bone with the burr 8 while moving it laterally across the internal opening 10 between the guide wires 32a, 32b. For example, a surgeon may move the burr laterally between the guide wires across the internal area 10 as shown by the arrows 44 in FIG. 4, then move the track set 24 a small distance longitudinally along the frame 4, then move the burr 8 across the internal area 10 again so that the burr cuts successive grooves in the bone 22 across the internal area 10. By sliding the track set 24 longitudinally along the frame 4 the surgeon can also move the burr 8 longitudinally along the internal area 10. When the surgeon moves the burr 8 longitudinally, the movement is guided by the guide wires 32a, 32b, slideably affixed to the frame 4. The skirt 26 is located on the shaft 33 so that when the burr 8 cuts to the required depth to countersink the plate 6 the skirt 26 encounters the track set 24 and is prevented from going deeper. In this manner, after several passes, a uniform layer is removed from the bone 22 throughout the entire internal area 10, creating a recess 46 for countersinking the plate 6.

Once the template 2 has been used to guide the burr 8 to form the recess 46 completely the template 2 is removed and the plate 6 is installed and fastened in place in the recess 46 by use of appropriate fasteners such as screws 50, as shown in FIG. 5

In another embodiment, as shown in FIG. 6, a frame 4′ itself may guide and limit the depth reachable by the cutter without a separate track set. The surgeon may use a cutting burr 8 with a skirt 43 which is wider than the internal area 10. Such a skirt 43 may completely bridge the internal area 10, and thus at all times when the burr 8 is in use at least some part of the skirt 43 rests on the top surface 42 of the frame 4′. The frame 4′ has a thickness 45 to cooperate with the location of the skirt 43 on the shaft of the burr 8 to limit the depth to which the burr 8 can reach. In this way, the frame 4′ may serve to define the length, width, and depth of the area of bone removed in order to countersink the plate. The surgeon guides the burr 8 throughout the internal area 10, the skirt 43 resting on the frame 4′ at all times, so that a layer of the bone 22 is uniformly and safely removed from the entire internal area 10.

Surgical plates may be used to interconnect two individual bones, as when fusing two or more vertebrae, for example. Plates may also be used to interconnect sections of a fractured bone. A fracture may transform one bone into two or more fragments or sections, which may be interconnected with one or more plates. Plates may also be used to interconnect or reinforce bony areas which are not fully fragmented by a fracture, such as sections of a skull surrounding a fracture. In all occasions, plates bridge bony areas and thus hold them in a rigid or semi rigid relationship so that healing may occur. Consequently, a cavity in which a plate may be countersunk must bridge the bony areas to be interconnected, as shown in FIG. 3.

The avoidance of dysphagia and other complications caused by the profile of a protruding plate is one reason to countersink a plate. Other reasons exist as well. For example, a plate may be composed of biologically active material, and may be designed to interact biologically, chemically, or cellularly with bone or other tissue, and countersinking or imbedding the plate may enhance or facilitate this interaction. Other reasons to countersink a plate may also exist or may be apparent to one of skill in the art.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.
What is claimed is:

1. A method of installing an implant in at least one bone, comprising:
(a) providing an implant having a predetermined shape and size;
(b) providing a template having a track for guiding a cutter in three dimensions;
(c) placing said template on said at least one bone and guiding said cutter with said track using said cutter to form a cavity with a shape and size determined by said template; and
(d) thereafter, affixing said implant to said at least one bone within said cavity.

2. The method of claim 1 wherein said implant is a surgical plate.

3. The method of claim 1 wherein said at least one bone is at least one vertebra.

4. The method of claim 3 wherein said at least one vertebra is at least one cervical vertebra.

5. The method of claim 1 further comprising the step of removeably affixing said template to said at least one bone.

6. A method of interconnecting at least two bony areas comprising:
(a) providing a surgical plate having a length, width, and a thickness;
(b) creating a cavity in said at least two bony areas, said cavity bridging said bony areas; said cavity having a length and a width substantially the same as said length and said width of said plate and having a depth substantially the same as said thickness of said plate;
(c) fitting said plate within said cavity; and
(d) fastening said plate to said at least two bony areas.

7. The method of claim 6 wherein said implant is a surgical plate.

8. The method of claim 6 wherein said at least one bone is at least one vertebra.

9. The method of claim 8 wherein said at least one vertebra is at least one cervical vertebra.

10. A method of affixing a surgical plate to at least one bone comprising:
(a) providing a plate having a length, a width, and a thickness;
(b) placing a template on said at least one bone, said template defining an internal area having a length and a width substantially the same as said plate, and said template further having means for guiding a cutter in three dimensions;
(c) associating a cutter with said means for guiding and forming a cavity with said cutter, said cavity having a length and width substantially the same as said length and said width of said plate and having a depth substantially the same as said thickness of said plate; and
(d) affixing said plate to said at least one bone within said cavity.

11. The method of claim 10 wherein said at least one bone is at least one vertebra.

12. The method of claim 11 wherein said at least one vertebra is at least one cervical vertebra.

13. The method of claim 10 further comprising the step of removably affixing said template to said at least one bone.

14. An apparatus for guiding the removal of bone in order to countersink an implant, said implant having a length, a width and a thickness, said apparatus comprising:
(a) a template, said template including a frame defining an internal area having a length and a width related to the length and width of said implant.

15. The apparatus of claim 14 which said implant is a surgical plate.

16. The apparatus of claim 14, further comprising a cutter associated with said frame.

17. The apparatus of claim 14, said frame further having means for limiting the depth reachable by a cutter associated with said template.

18. The apparatus of claim 14 wherein said template further includes a track set for guiding a cutter in three dimensions.

19. The apparatus of claim 14 wherein said frame has two side pieces interconnecting two end pieces, and wherein said track includes guide channels extending along each said side piece and a slotted rail slideably affixed to said guide channels, said slotted rail defining a depth reachable by said cutter.
